(19) United States
(12) Patent Application Publication
(10) Pub. No.: US 2023/0199338 A1
(43) Pub. Date: Jun. 22, 2023

(54) RADIATION IMAGING APPARATUS AND METHOD OF CONTROLLING THE SAME, AND STORAGE MEDIUM

(71) Applicant: CANON KABUSHIKI KAISHA, Tokyo (JP)

(72) Inventors: JUNKI ADACHI, Tokyo (JP); Kazumasa Matsumoto, Tokyo (JP)

(21) Appl. No.: 18/064,212

(22) Filed: Dec. 9, 2022

(30) Foreign Application Priority Data
Dec. 20, 2021 (JP) 2021-206267
Sep. 28, 2022 (JP) 2022-155294

(51) Int. Cl. H04N 25/60; H04N 25/78; H04N 25/42

(52) U.S. Cl. CPC H04N 25/60; H04N 25/78; H04N 25/42; A61B 6/461

(57) ABSTRACT

A radiation imaging apparatus comprises a plurality of pixels arranged in a matrix pattern, each pixel including a signal generation portion configured to generate a signal based on accumulated charges and a holding portion configured to hold a signal output from the signal generation portion, a switch configured to commonly connect a predetermined number of pixels of the plurality of pixels to add the signals from the predetermined number of pixels, and a readout circuit configured to read out accumulation signals held in the holding portion over a plurality of times while changing an addition region by using the switch and performing pixel addition and obtain a plurality of images different in pixel addition count with respect to one time of charge accumulation.

### BACKGROUND

### Field of Disclosure

The present disclosure generally relates to radiography, and more particularly it relates to a technique of obtaining correction images in a radiation imaging apparatus.

### Description of Related Art

In the field of radiation imaging apparatuses, in order to achieve an improvement in resolution, a reduction in volume, suppression of image deformation, and the like, a large-area flat-panel radiation imaging apparatus of an equal-magnification optical system type using photoelectric conversion elements has been widely used.

Japanese Patent No. 4551588 discloses a scheme of reading out signals at a high frame rate even with a reduction in resolution by reading out, as addition signals, signals obtained by adding outputs from pixels in an imaging region, in addition to performing a standard operation of an imaging apparatus that reads out signals from all the pixels in the imaging region.

Japanese Patent Laid-Open No. 2014-30151 discloses a technique of correcting a radiation image using correction image data by averaging a plurality of added dark current images obtained by imaging without irradiation of radiation in order to suppress the influence of fixed pattern noise (FPN).

The scheme of reading out, as addition signals, signals obtained by adding outputs from pixels in the imaging region is sometimes provided with a plurality of types (for example, four types) of pixel addition modes such as 1×1, 2×2, 4×4, and 8×8 pixel addition modes with respect to one imaging mode. Conventionally, in such a case, imaging is performed a plurality of different times (for example, four times) for each pixel addition mode, and correction images are generated in different pixel addition modes based on a plurality of frames of images obtained in the respective modes. Accordingly, it takes relatively long time to generate a correction image.

### SUMMARY

The present disclosure improves on the process for generating a correction image and enables a radiation imaging apparatus to more efficiently obtain a correction image for image quality improvement.

According to a first aspect of the present disclosure, there is provided a radiation imaging apparatus comprising: a plurality of pixels arranged in a matrix pattern, each pixel including a signal generation portion configured to generate a signal based on accumulated charges and a holding portion configured to hold a signal output from the signal generation portion; a switch configured to commonly connect a predetermined number of pixels of the plurality of pixels to add the signals from the predetermined number of pixels; and a readout circuit configured to read out accumulation signals held in the holding portion over a plurality of times while changing an addition region by using the switch and performing pixel addition and obtain a plurality of images different in pixel addition count with respect to one time of charge accumulation.

According to a second aspect of the present disclosure, there is provided a method of controlling a radiation imaging apparatus including a plurality of pixels arranged in a matrix pattern, each pixel including a signal generation portion configured to generate a signal based on accumulated charges and a holding portion configured to hold a signal output from the signal generation portion and a switch configured to commonly connect a predetermined number of pixels of the plurality of pixels to add the signals from the predetermined number of pixels, the method comprising: reading out accumulation signals held in the holding portion over a plurality of times while performing pixel addition in a direction to sequentially enlarge an addition region by using the switch and obtaining a plurality of images different in pixel addition count with respect to one time of charge accumulation.

According to a third aspect of the present disclosure, there is provided a non-transitory computer-readable storage medium storing a program for executing a method of controlling a radiation imaging apparatus including a plurality of pixels arranged in a matrix pattern, each pixel including a signal generation portion configured to generate a signal based on accumulated charges and a holding portion configured to hold a signal output from the signal generation portion and a switch configured to commonly connect a predetermined number of pixels of the plurality of pixels to add the signals from the predetermined number of pixels, the method comprising: reading out accumulation signals held in the holding portion over a plurality of times while performing pixel addition in a direction to sequentially enlarge an addition region by using the switch and obtaining a plurality of images different in pixel addition count with respect to one time of charge accumulation.

Further features of the present disclosure will become apparent from the following description of exemplary embodiments with reference to the attached drawings.

### BRIEF DESCRIPTION OF THE DRAWINGS

FIG. 1 is an exemplary circuit diagram for explaining the schematic circuit of one pixel in a radiation imaging apparatus according to the first embodiment of the present disclosure;

FIG. 2A is an exemplary circuit diagram for explaining the schematic arrangement of the pixel array of the radiation imaging apparatus;

FIG. 2B is an exemplary circuit diagram for explaining the schematic arrangement of a signal readout portion;

FIG. 3A is an example of a pixel addition mode setting table;

FIG. 3B is a view showing a pixel group in the 1×1 pixel addition mode;

FIG. 3C is a view showing a pixel group in the 2×2 pixel addition mode;

FIG. 3D is a view showing a pixel group in the 4×4 pixel addition mode;

FIG. 3E is a view showing a pixel group in the 8×8 pixel addition mode;

FIG. 4 is a schematic view for explaining the arrangement of a radiation imaging system according to an embodiment of the present disclosure;

FIG. 5 is a timing chart showing the operation of reading out signals while switching the pixel addition modes;

FIGS. 6A and 6B are flowcharts showing the operation of generating correction images in the different pixel addition modes;

FIG. 7 is an exemplary circuit diagram for explaining the schematic circuit of one pixel in a radiation imaging apparatus according to the second embodiment of the present disclosure;

FIG. 8 is a timing chart showing the operation of reading out signals while switching sensitivity modes and the pixel addition modes; and

FIGS. 9A and 9B are flowcharts showing the operation of generating correction images in the different sensitivity modes and the different pixel addition modes.

### DESCRIPTION OF THE EMBODIMENTS

Hereinafter, embodiments will be described in detail with reference to the attached drawings. Note, the following detailed description provides details sufficient to both make and use the embodiments, but the embodiments are not intended to limit the scope of the claims. Multiple features are described in the various embodiments, but no embodiment requires all such features, and multiple such features may be combined as appropriate. Furthermore, in the attached drawings, the same reference numerals are given to the same or similar components, processes or configurations, and redundant description thereof is omitted.

### First Embodiment

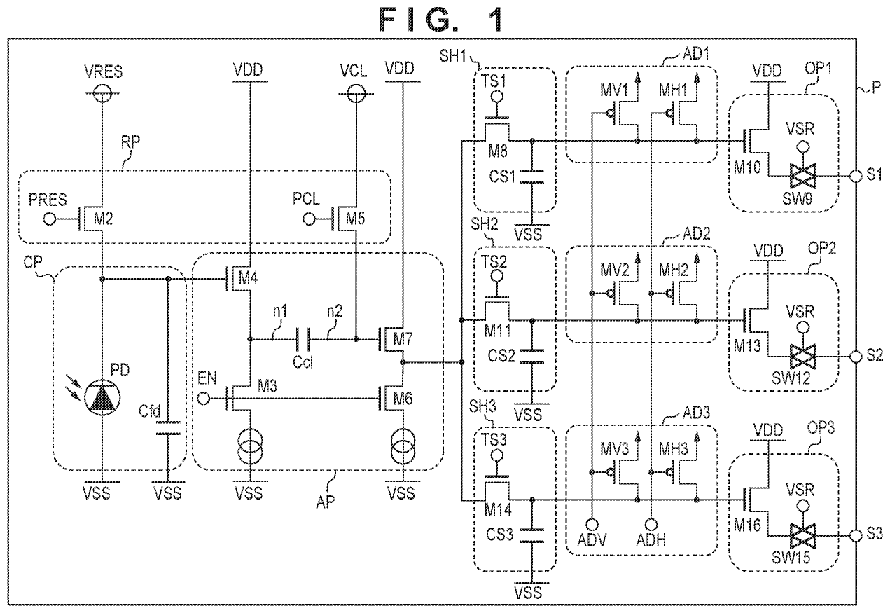

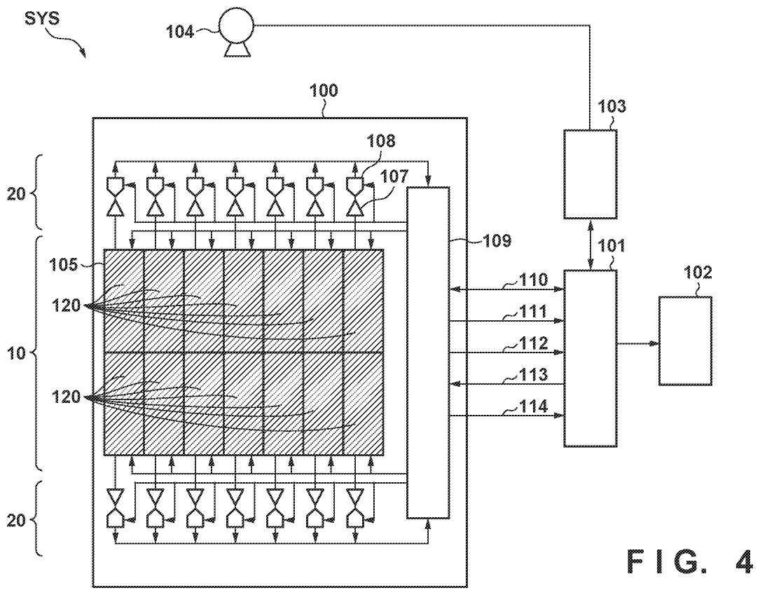

FIG. 1 is an exemplary circuit diagram for explaining the schematic arrangement of one pixel P in a radiation imaging apparatus 100 (see FIG. 4) according to the first embodiment of the present disclosure. A pixel P includes a conversion portion CP, an amplification portion AP, a reset portion RP, holding portions SH1 to SH3, pixel addition portions AD1 to AD3, and output portions OP1 to OP3. In the following case, these components are formed by circuits. For example, the conversion portion CP is formed by a conversion circuit. Please note that VDD denotes a power supply voltage in the pixel P exemplary circuit, and VSS denotes the ground in the pixel P exemplary circuit.

In the conversion portion CP, a photodiode PD generates charges in an amount corresponding to radiation, and the amplification portion AP outputs the voltage generated by an FD capacitor (floating diffusion capacitor) Cfd in accordance with the generated amount of charges.

In the amplification portion AP, an enable signal EN is activated to input a voltage from an amplification transistor M4, which receives a voltage from the conversion portion CP, to an amplification transistor M7, and the input voltage is then amplified and output from the amplification transistor M7.

The reset portion RP resets (initializes) the charges in the photodiode PD upon activation of a reset signal PRES. When a clamp signal PCL is activated, a clamp voltage VCL as a predetermined potential is input to an output terminal n2 of a clamp capacitor Cc1.

The conversion portion CP and the amplification portion AP constitute a signal generation portion that generates a signal based on the charges generated and accumulated in the photodiode PD. This signal is called an accumulation signal. Resetting of the signal generation portion is performed by resetting the potential of the photodiode PD (a photoelectric conversion element) and the potential of the output terminal n2 of the clamp capacitor Cc1 in the above manner.

The holding portion SH1, the holding portion SH2, and the holding portion SH3 each are a sample hold circuit capable of holding the pixel signal output from the amplification portion AP. Each pixel P has a plurality of holding portions. In this embodiment, each pixel P has three holding portions. The output portion OP1, the output portion OP2, and the output portion OP3 corresponding to the holding portions SH1 to SH3 amplify and output the pixel signals respectively held in a holding capacitor CS1, a holding capacitor CS2, and a holding capacitor CS3. The output portion OP1 has a signal amplification transistor M10 and a switch SW9 for inputting the vertical scanning signal VSR. The output portion OP2 has a signal amplification transistor M13 and a switch SW12 for inputting the vertical scanning signal VSR. The output portion OP3 has a signal amplification transistor M16 and a switch SW15 for inputting the vertical scanning signal VSR. When pixel signals corresponding to the holding portions SH1 to SH3 are accumulation signals, they will be referred to as an accumulation signal S1, an accumulation signal S2, and an accumulation signal S3.

After sampling and holding by the capacitors CS1, CS2, and CS3, transfer transistors M8, M11, and M14 are turned off, and the capacitors CS1, CS2, and CS3 are thus disconnected from the amplification portion AP on the preceding stage. For this reason, the pixel signal (the accumulation signal or the reset signal) held by one time of charge accumulation can be read out nondestructively a plurality of times until it is sampled and held again.

The pixel addition portion AD1 includes a pixel addition switch MV1 in the vertical direction and a pixel addition switch MH1 in the horizontal direction. The pixel addition switches are commonly connected to the holding capacitors CS1 of adjacent pixel circuits (adjacent pixels) in the vertical and horizontal directions. Pixel addition is performed by conductively connecting the holding capacitors CS1 of the adjacent pixels using the pixel addition switches MV1 and MH1 in accordance with pixel addition control signals ADV and ADH. This makes it possible to add the voltages accumulated in the plurality of holding capacitors CS1 conductively connected to each other, in other words, average the potentials, and output the resultant potential.

Likewise, the pixel addition portion AD2 can add the charges accumulated in the holding capacitors CS2 of the adjacent pixels, and the pixel addition portion AD3 can add and output the voltages accumulated in the holding capacitors CS3 of the adjacent pixels.

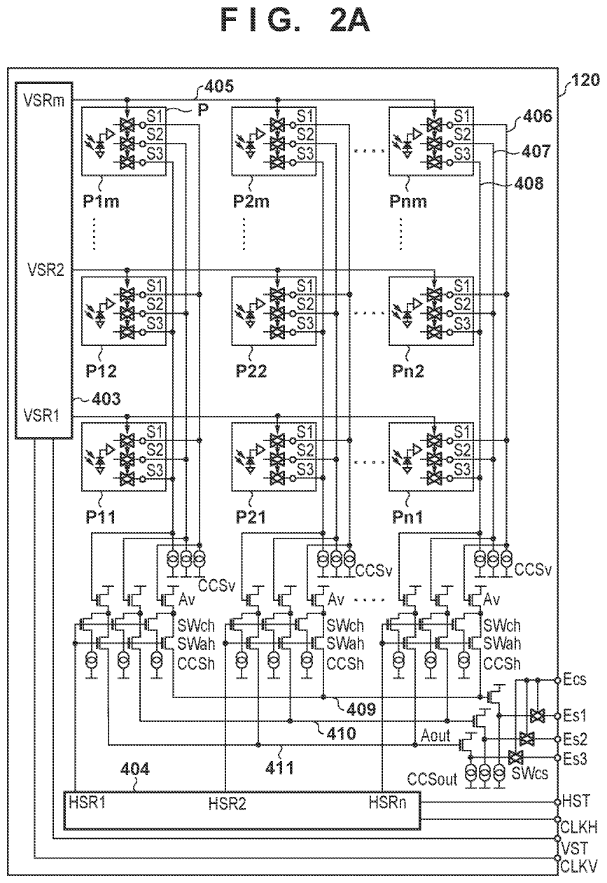

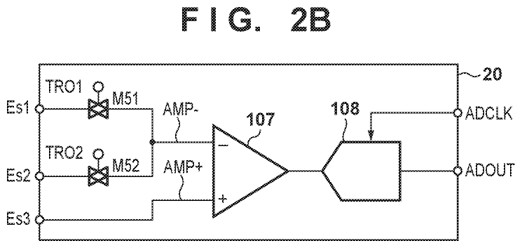

A pixel array 120 and a signal readout portion 20 of the radiation imaging apparatus 100 according to this embodiment will be described next with reference to FIGS. 2A and 2B. The signal readout portion 20 reads out signals from the pixel array 120. The pixel array 120 having the pixels P arranged in a two-dimensional array pattern (a matrix pattern of n×m pixels). FIG. 2A is an exemplary circuit diagram for explaining the schematic arrangement of the pixel array 120 of the radiation imaging apparatus 100 according to the embodiment.

The pixel array 120 includes the plurality of pixels P, a vertical scanning circuit 403 for driving each pixel P, and a horizontal scanning circuit 404 for reading out a signal from each pixel P. The pixel array 120 also includes a terminal Es1 for reading out the pixel signal held in the holding capacitor CS1 of each pixel P, a terminal Es2 for reading out the pixel signal held in the holding capacitor CS2, and a terminal Es3 for reading out the pixel signal held in the holding capacitor CS3.

The outputs of amplification transistors Av are respectively connected to analog signal lines 409 to 411 via transfer transistors SWah that are set in the conductive state in response to horizontal scanning signals HSR from the horizontal scanning circuit 404. The voltages obtained by amplifying voltages from the analog signal lines 409 to 411 by amplification transistors Aout are output from the terminals Es1, Es2, and Es3. The pixel signals S1, S2, and S3 are sequentially read out from each pixel P by an X-Y address method in accordance with control signals input from a control portion 109 (to be described later) (see FIG. 4).

The signal readout portion 20 of the radiation imaging apparatus 100 according to this embodiment will be described next with reference to FIG. 2B. FIG. 2B is an exemplary circuit diagram for explaining the schematic arrangement of the signal readout portion 20. The signal readout portion 20 includes a signal amplification portion 107 including, for example, a differential amplifier, and an AD conversion portion 108 that performs AD (analog-to-digital) conversion.

The pixel signal S3 from the terminal Es3 is input to a noninverting input terminal AMP+ of the signal amplification portion 107. The pixel signal S1 from the terminal Es1 is input to an inverting input terminal AMP− via a switch M51. The pixel signal S2 from the terminal Es2 is input to the inverting input terminal AMP− via a switch M52. The switches M51 and M52 are controlled by a control signal TRO1 and a control signal TRO2 such that one of signals from terminal Es1 and the terminal Es2 is input to the inverting input terminal AMP-.

The signal amplification portion 107 amplifies the difference (subtraction result) between a signal from the terminal Es1 and a signal from the terminal Es3 or the difference between a signal from the terminal Es2 and a signal from the terminal Es3. The AD conversion portion 108 AD-converts the difference based on a clock signal input via a terminal ADCLK. With this arrangement, image data (digital data) is obtained from the pixel array 120 and output to the control portion 109 (to be described later) via a terminal ADOUT.

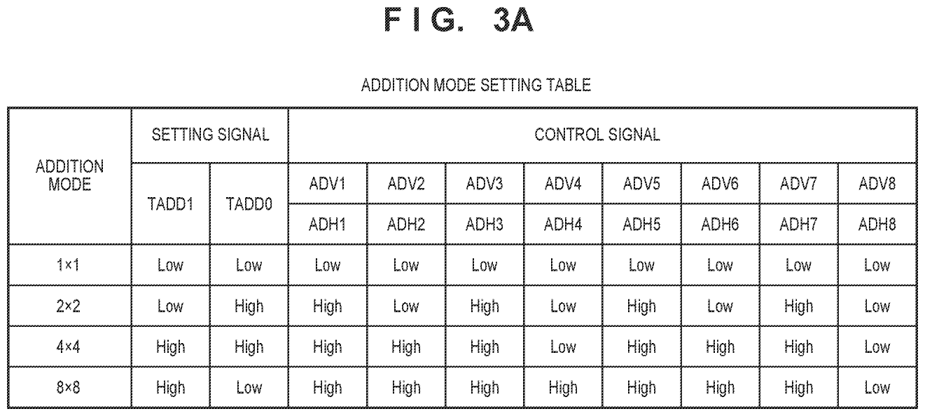

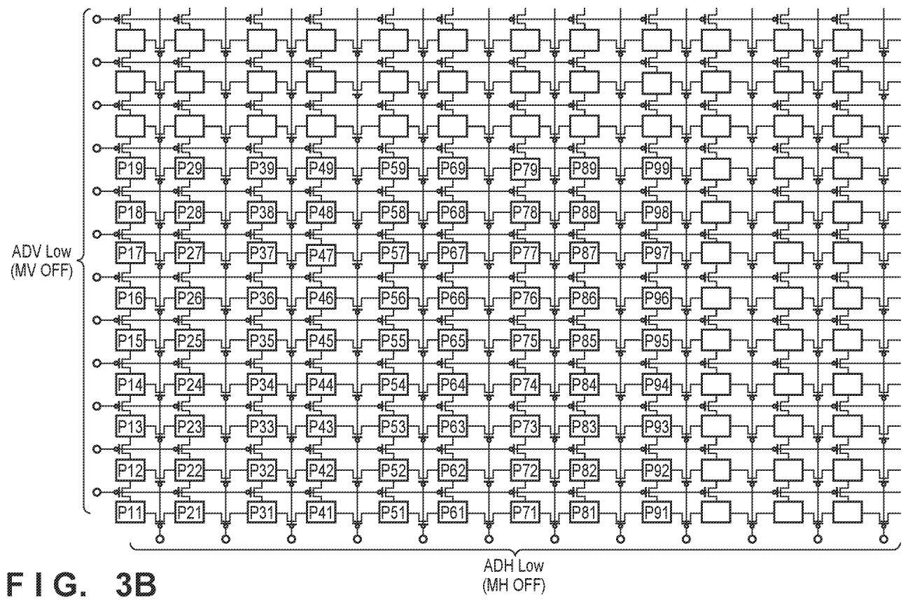

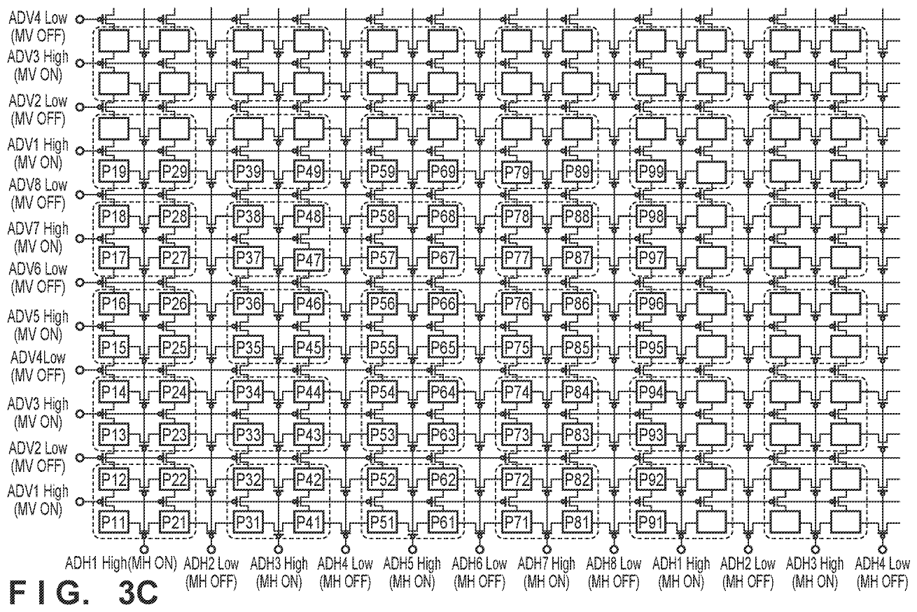

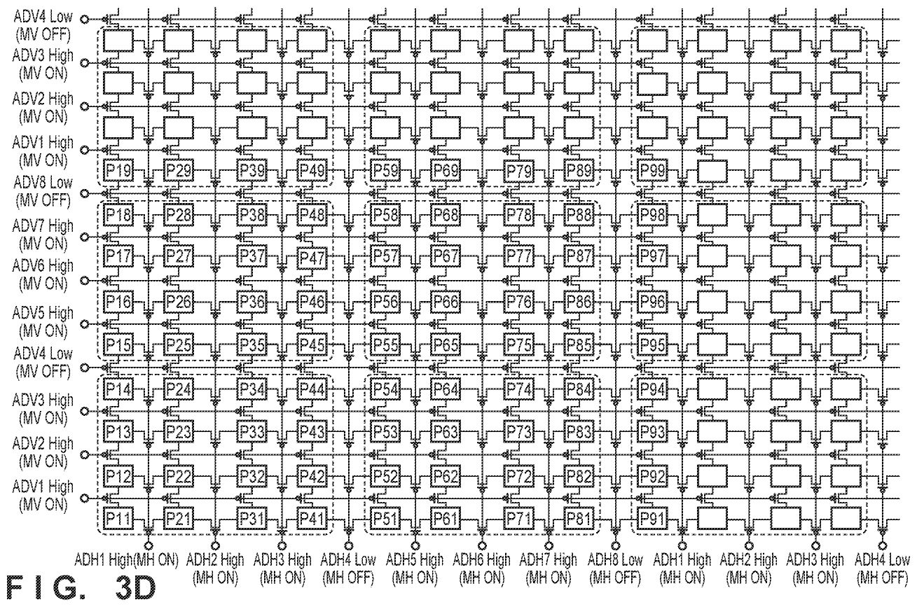

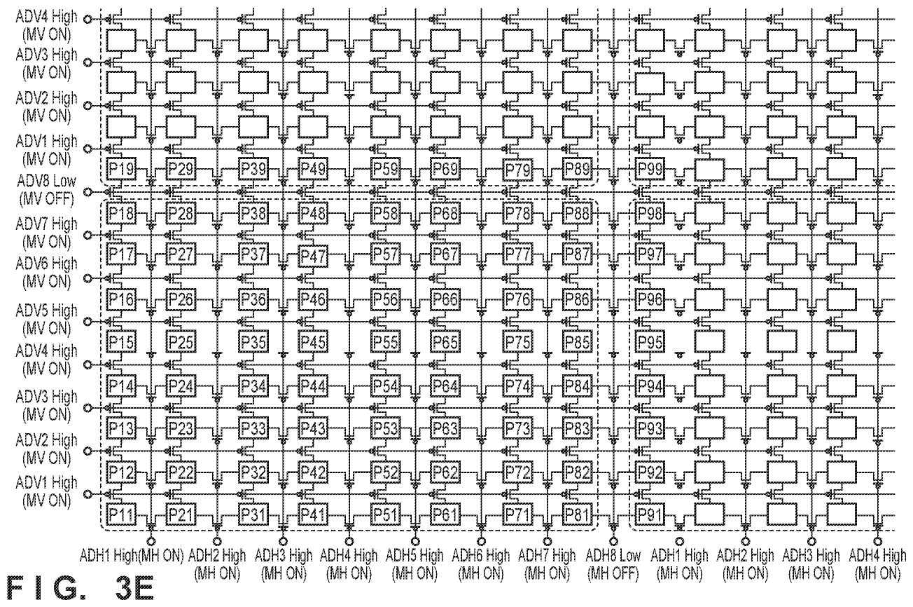

Pixel addition in this embodiment will be described next with reference to FIGS. 3A to 3E.

FIG. 3A is an example of an addition mode setting table indicating setting signals that are output by the control portion 109 of the radiation imaging apparatus 100 (to be described later) to the pixel array 120 to switch addition regions, that is, addition modes, for pixel addition and pixel addition control signals that are decoded inside the pixel array 120 in accordance with the setting signals. FIGS. 3B to 3E are conceptual diagrams showing pixel addition in accordance with this embodiment.

As described above, holding capacitors subjected to pixel addition include holding capacitors of three systems, namely, the holding capacitors CS1, CS2, and CS3, in one pixel. For pixel addition, the same addition circuit is formed in the holding capacitor of each system, and the three systems are subjected to addition processing in the same manner. In this case, pixel addition in the holding capacitor of one system will be described below.

In the addition mode table in FIG. 3A, TADD0 and TADD1 represent addition mode setting signals, ADV1 to ADV8 represent pixel addition control signals in the vertical direction, and ADH1 to ADH8 represent pixel addition control signals in the horizontal direction.

When a pixel addition control signal is in the “Low” state, the pixel addition switch controlled by the pixel addition control signal is set in the non-conductive state. When a pixel addition control signal is in the “High” state, the pixel addition switch controlled by the pixel addition control signal is set in the conductive state to form a pixel group, thereby adding the voltages accumulated in a plurality of holding capacitors in the pixel group, that is, averaging the potentials.

In this embodiment, in reading out image signals (to be described later), the signals are read out while pixel addition modes (a plurality of modes different in pixel addition count) are sequentially switched (sequentially enlarged) in order of 1×1, 2×2, 4×4, and 8×8. Accordingly, the addition mode setting table in FIG. 3A is formed with consideration that control signals for the pixel addition switches are switched without any error when the pixel addition modes are sequentially switched in order of 1×1, 2×2, 4×4, and 8×8. More specifically, in switching between preceding and succeeding addition modes, the hamming distances between codes of the addition mode setting signals TADD0 and TADD1 of two bits are set to 1.

P11 to P99 in FIGS. 3B to 3E represent pixels.

FIG. 3B shows that both TADD0 and TADD1 are at “Low”, and all the pixel addition control signals indicate that the addition switches are in the non-conductive state. This is the 1×1 addition mode, that is, in the state in which no pixel addition is performed.

FIG. 3C shows that TADD0 and TADD1 are respectively set at “High” and “Low”, the pixel addition control signals ADV1, ADV3, ADV5, ADV7, ADH1, ADH3, ADH5, and ADH7 are in the “High” state, and the remaining pixel addition control signals are in the “Low” state. The pixel addition switches corresponding to the “High” pixel addition control signals are set in the conductive state, and the potentials of four pixels surrounded by a broken line, for example, P11, P21, P12, and P22, are averaged and set in a 2×2 pixel addition state. Likewise, FIG. 3D shows an example of a 4×4 state, and FIG. 3E shows an example of an 8×8 state.

The vertical scanning circuit 403 for driving each pixel P at the time of pixel addition and the horizontal scanning circuit 404 for reading out a signal from each pixel P function to perform decimated scanning so as to read out, as one pixel, a pixel group obtained by addition in accordance with the pixel addition mode.

The radiation imaging apparatus 100 and a radiation imaging system SYS including the pixel addition function according to this embodiment are formed by using the pixel array 120, the signal readout portion 20, and the pixel addition portions AD1, AD2, and AD3 like those described above.

The radiation imaging apparatus 100 and the radiation imaging system SYS according to this embodiment will be described next with reference to FIG. 4. FIG. 4 is a schematic view for explaining the schematic arrangements of the radiation imaging apparatus 100 and the radiation imaging system SYS according to the embodiment.

The radiation imaging system SYS includes the radiation imaging apparatus 100, a radiation generation apparatus 104 that generates radiation, an irradiation control portion 103, a signal processing portion 101 that performs image processing and system control, and a display portion 102 including a display. When performing radiation imaging, the signal processing portion 101 synchronously controls the radiation imaging apparatus 100 and the irradiation control portion 103. The radiation imaging apparatus 100 generates a signal based on radiation (X-rays, α-rays, β-rays, γ-rays, or the like) that has passed through a subject. After the signal processing portion 101 and the like perform predetermined processing for the signal, image data based on the radiation is generated. The image data is displayed as a radiation image on the display portion 102. The radiation imaging apparatus 100 includes an imaging panel 105 including an imaging region 10, the readout circuit 20 that reads out a signal from the imaging region 10, and the control portion 109 that controls each unit.

The imaging panel 105 is formed by tiling (two-dimensionally arranging) a plurality of pixel arrays 120 on a plate-shaped base, and the large imaging panel 105 is formed by this arrangement. A plurality of pixels P are arranged in each pixel array 120. The imaging region 10 includes the plurality of pixels P that are arranged such that a row and a plurality of columns are formed by the plurality of pixel arrays 120. In addition, an arrangement obtained by tiling the plurality of pixel arrays 120 to form 7 columns×2 rows is exemplified here. However, the number of columns and the number of rows are not limited to this arrangement.

The control portion 109 performs communication of a control command and communication of a synchronization signal with, for example, the signal processing portion 101, and outputs image data to the signal processing portion 101. In addition, the control portion 109 controls the imaging region 10 or each unit and, for example, sets the reference voltage of each pixel array 120 and performs driving control of each pixel and operation mode control. The control portion 109 also synthesizes one frame image data using the image data (digital data) of each pixel array 120, which is A/D-converted by the A/D conversion portion 108 of the readout circuit 20, and outputs the data to the signal processing portion 101. The control unit 109 may be formed by a processor such as a CPU and memories such as a RAM and a ROM. The operation of the radiation imaging apparatus 100 to be described later may be executed by executing a program stored in the memory by the processor of the control unit 109. Alternatively, the control portion 109 may be formed by a dedicated circuit such as an ASIC (Application Specific Integrated Circuit).

Transmission/reception of a control command or a control signal and image data is performed between the control portion 109 and the signal processing portion 101 via various kinds of interfaces. The signal processing portion 101 outputs setting information such as an operation mode and various kinds of parameters or imaging information to the control portion 109 via a control interface 110. In addition, the control portion 109 outputs device information such as the operating state of the radiation imaging apparatus 100 to the signal processing portion 101 via the control interface 110. The control portion 109 also outputs image data obtained by the radiation imaging apparatus 100 to the signal processing portion 101 via an image data interface 111. The control unit 109 also notifies the signal processing portion 101, using a READY signal 112, that the radiation imaging apparatus 100 has changed to an imaging enable state. In addition, the signal processing portion 101 notifies the control portion 109 of the timing of the start of radiation irradiation using a synchronization signal 113 in response to the READY signal 112 from the control portion 109. An irradiation permission signal 114 is a signal that notifies the signal processing portion 101 that the imaging panel 105 is executing accumulation. When the irradiation permission signal 114 is in the enable state, the signal processing portion 101 outputs a control signal to the irradiation control portion 103 and causes it to start radiation irradiation.

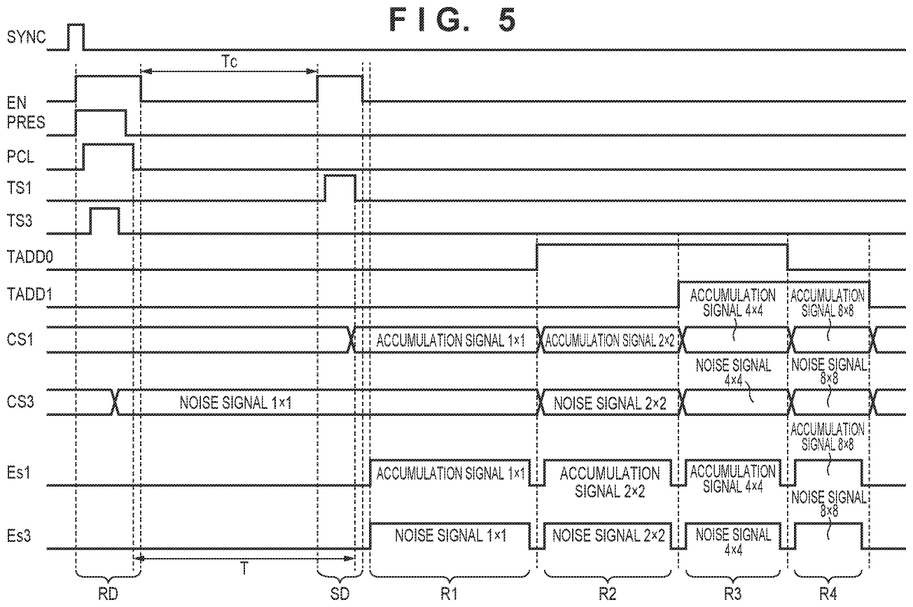

FIG. 5 is a timing chart showing an example of a driving method of continuously reading out correction images different in pixel addition group by one time of accumulation while sequentially switching the resolution from the high resolution to the low resolution in the radiation imaging apparatus 100. The control portion 109 executes this method by controlling the operation of each component of the radiation imaging apparatus 100. The radiation imaging apparatus 100 captures a moving image constituted by a plurality of frame images. Referring to the timing chart in FIG. 5, assume that a signal from the output terminal Es1 for the S1 signal from the pixel array 120 is always selected as the AMP− signal from the signal amplification portion 107 of the signal readout portion 20.

An accumulation period T indicates an accumulation period corresponding to a frame image. During the accumulation period T, when radiation is applied, the control portion 109 transmits the irradiation permission signal 114 to the signal processing portion 101 to notify it that X-rays can be applied. When a correction dark image (dark signal) is obtained, image capturing is performed without the transmission of the irradiation permission signal 114 and without radiation irradiation. Referring to FIG. 5, the signal names “SYNC” to “TADD1” represent the levels of signals. “CS1” and “C53” represent signals held by the holding capacitors CS1 and CS3. “Es1” and “Es3” represent periods to read out signals from the pixel array 120 to the readout circuit 20. The control unit 109 executes the signal readout operation while “Es1” and “Es3” are at high level.

An imaging mode is set before imaging capturing. Upon detecting the leading edge of a pulse of the signal SYNC, the control portion 109 starts driving to generate a frame image. The signal SYNC can be either an external synchronization signal or an internal synchronization signal, and an external synchronization signal SYNC is used in this embodiment. RD and SD respectively represent reset driving and sample hold driving. The reset driving is driving for the operation of resetting the conversion portion CP and the amplification portion AP and sampling and holding the clamp voltage VCL at the time of resetting as a noise signal in the holding capacitor CS3. The sample hold driving is driving for sampling and holding the accumulation signal which has changed from the clamp voltage VCL in the accumulation period T in the holding capacitor CS1.

The reset driving RD will be described. Upon detecting the leading edge of a pulse of the signal SYNC, the control portion 109 starts driving for the generation of a frame image. The control portion 109 activates the enable signal EN and the reset signal PRES. As a result, a reset voltage VRES as a predetermined potential is supplied to the photodiode PD to reset the charges in the photodiode PD. The clamp signal PCL is then activated. This inputs the clamp voltage VCL as a predetermined potential to the output terminal n2 of the clamp capacitor Cc1.

A control signal TS3 is temporarily activated until the clamp signal PCL is inactivated. This transfers the clamp voltage VCL as a noise signal to the holding capacitor CS3. Upon temporarily activating the control signal TS3, the control portion 109 inactivates the reset signal PRES and then inactivates the clamp signal PCL. This starts the accumulation period T. The control portion 109 inactivates the enable signal EN. This ends the reset driving RD.

The sample hold driving SD will be described next. After the elapse of a period Tc since the inactivation of the enable signal EN, the control portion 109 activates the enable signal EN and then temporarily activates a control signal TS1. With this operation, the accumulation signal S1 accumulated in the period T is transferred and held in the holding capacitor CS1 (that is, the accumulation signal is sampled). Upon completion of the sampling, the control portion 109 inactivates the enable signal EN. This ends the sample hold driving SD in a frame period F1.

Readout driving for continuously reading out images while switching the pixel addition modes in order of 1×1, 2×2, 4×4, and 8×8 from a period R1 to a period R4 will be described next with reference to FIG. 5.

At the time of the end of the sample hold driving SD, the addition mode is set to the 1×1 addition mode with TADD0 and TADD1 being both at “Low”, and the accumulation signal S1 and the noise signal S3 are respectively held in the holding capacitor CS1 and the holding capacitor CS3 while each pixel is independently separated.

Upon completion of the sample hold driving SD, the control portion 109 starts reading out images from the pixels in the entire imaging region while maintaining the state of the 1×1 addition mode, that is, keeping both TADD0 and TADD1 at “Low”.

The control portion 109 reads out the held accumulation signal S1 and noise signal S3 via the signal paths (differential signal paths) of the pixel signals of two systems in the pixel array 120 and outputs them to the signal amplification portion 107 of the signal readout portion 20. The signal amplification unit 107 that has received the output from the pixel array 120 outputs a signal obtained by calculating the difference between the accumulation signal S1 and the noise signal S3.

The AD conversion portion 108 converts this output signal into digital data and supplies it to the control portion 109. The control portion 109 controls the vertical scanning circuit 403 and the horizontal scanning circuit 404 to sequentially switch selected pixels one by one and generate digital data for the generation of a 1×1 image in the period R1, thereby generating an image based on the accumulation signal. An image generated by reading out an accumulation signal in the 1×1 addition mode is called a 1×1 accumulation image.

The control portion 109 then switches TADD0 from “Low” to “High” to switch the pixel addition mode to the 2×2 pixel addition mode.

The control portion 109 generates an image based on an accumulation signal in the 2×2 addition mode in a period R2 by controlling the vertical scanning circuit 403 and the horizontal scanning circuit 404 so as to sequentially switch selected pixels for each two pixels (a predetermined count) in accordance with the 2×2 pixel addition state. An image generated by reading out an accumulation signal in the 2×2 addition mode is called a 2×2 accumulation image.

The control portion 109 then switches TADD1 from “Low” to “High” to switch the pixel addition mode to the 4×4 pixel addition mode.

The control portion 109 generates an image based on an accumulation signal in the 4×4 addition mode in a period R3 by controlling the vertical scanning circuit 403 and the horizontal scanning circuit 404 so as to sequentially switch selected pixels for each four pixels in accordance with the 4×4 pixel addition state. An image generated by reading out an accumulation signal in the 4×4 addition mode is called a 4×4 accumulation image.

The control portion 109 then switches TADD0 from “High” to “Low” to switch the pixel addition mode to the 8×8 pixel addition mode.

The control portion 109 generates an image based on an accumulation signal in the 8×8 addition mode in a period R4 by controlling the vertical scanning circuit 403 and the horizontal scanning circuit 404 so as to sequentially switch selected pixels for each eight pixels in accordance with the 8×8 pixel addition state. An image generated by reading out an accumulation signal in the 8×8 addition mode is called an 8×8 accumulation image.

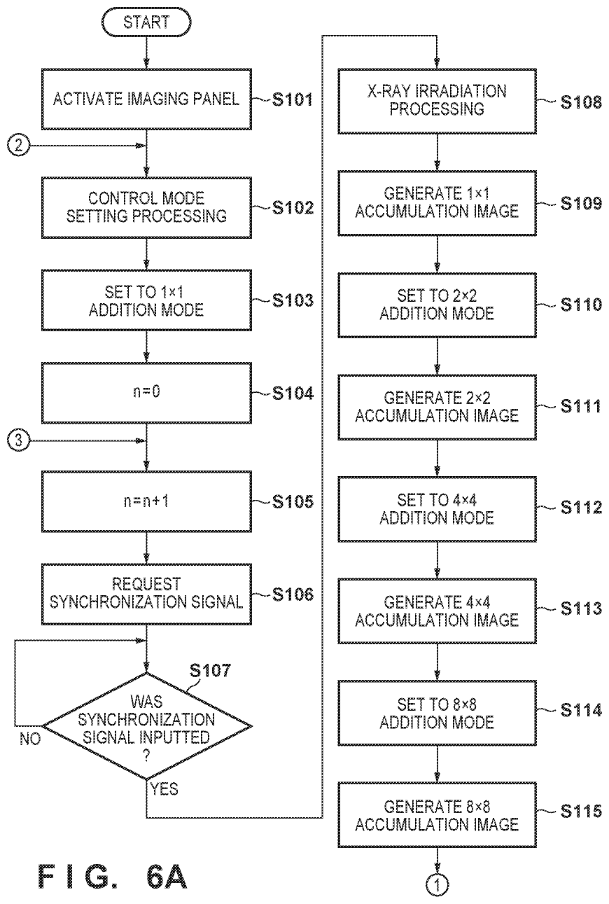

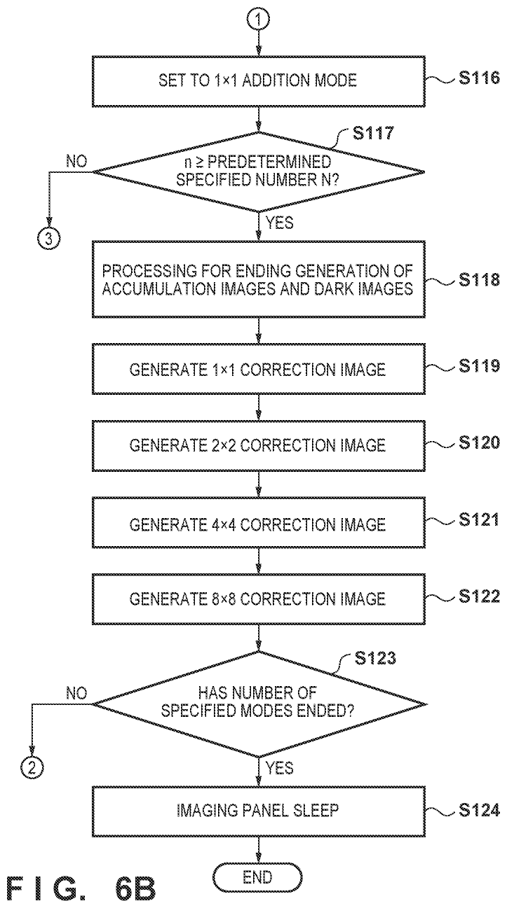

FIGS. 6A and 6B are flowcharts showing an example of processing of the radiation imaging apparatus 100 from the accumulation of images in synchronism with a synchronization signal to the generation of correction images in the different pixel addition modes, namely, the 1×1, 2×2, 4×4, and 8×8 pixel addition modes.

In step S101, the control portion 109 activates the imaging panel 105 to make a transition to an imaging enable state.

In step S102, the signal processing portion 101 issues a control command to the control portion 109 of the radiation imaging apparatus 100 via the control interface 110 to transfer information indicating that the correction image generation mode is set, together with information indicating the type of correction image, an accumulation time, an imaging count N, and the like. Upon receiving the control command, the control portion 109 sets a control mode for the radiation imaging apparatus 100, including a correction image generation sequence, permission/inhibition of X-ray irradiation, and an accumulation time.

In step S103, the control portion 109 sets the pixel addition mode of the pixel array 120 of the imaging panel 105 to the 1×1 addition mode as a default mode.

In step S104, an imaging counter (“n”) inside the control portion 109 is reset to 0.

In step S105, the control portion 109 increments the imaging counter to make the radiation imaging apparatus 100 prepare for imaging.

In step S106, the control portion 109 notifies the signal processing portion 101 via the control interface 110 that imaging is prepared and issues a command to request a synchronization signal. The signal processing portion 101 determines the status of the system and outputs a synchronization signal when the system is set in the imaging enable state.

In step S107, the control portion 109 waits for the input of the synchronization signal pulse SYNC sent as the synchronization signal 113 from the signal processing portion 101. When the synchronization signal pulse SYNC is input, the process makes a transition to step S108.

In step S108, the control portion 109 performs the reset driving RD and performs X-ray irradiation processing upon completion of the reset driving RD. When accumulating X-ray irradiation images, the control portion 109 notifies the signal processing portion 101 with the irradiation permission signal 114 that X-ray irradiation is permitted during the accumulation period T, thus accumulating images by irradiation with X-rays. When obtaining a dark image, the control portion 109 performs image accumulation without sending the irradiation permission signal 114 and without irradiation. After the elapse of the accumulation period T, the control portion 109 performs the sample hold driving SD and completes the X-ray irradiation processing.

In step S109, the control portion 109 reads out an accumulation signal in the 1×1 pixel addition mode, generates a 1×1 accumulation image, and saves the generated accumulation image in a storage portion (not shown) inside the radiation imaging apparatus 100.

In step S110, the control portion 109 changes the pixel addition mode of the pixel array 120 to the 2×2 pixel addition mode.

In step S111, the control portion 109 reads out an accumulation signal in the 2×2 pixel addition mode, generates a 2×2 accumulation image, and saves the generated accumulation image in the storage portion (not shown) inside the radiation imaging apparatus 100.

In step S112, the control portion 109 changes the pixel addition mode of the pixel array 120 to the 4×4 pixel addition mode.

In step S113, the control portion 109 reads out an accumulation signal in the 4×4 pixel addition mode, generates a 4×4 accumulation image, and saves the generated accumulation image in the storage portion (not shown) inside the radiation imaging apparatus 100.

In step S114, the control portion 109 changes the pixel addition mode of the pixel array 120 to the 8×8 pixel addition mode.

In step S115, the control portion 109 reads out an accumulation signal in the 8×8 pixel addition mode, generates an 8×8 accumulation image, and saves the generated accumulation image in the storage portion (not shown) inside the radiation imaging apparatus 100.

In step S116, the control portion 109 sets the pixel addition mode of the pixel array 120 of the imaging panel 105 to the 1×1 pixel addition mode as the default mode.

In step S117, the control portion 109 determines whether the processing of generating accumulation images in specified N different addition modes set in the control mode setting processing in step S102 is completed. If the specified number of times of generation is completed, the control portion 109 executes the processing in step S118. If the specified number of times of generation is not completed, the control portion 109 repeats the accumulation image generation processing from step S105.

Upon completion of the accumulation image generation processing in steps S101 to S117, the control portion 109 stores N accumulation images in the 1×1, 2×2, 4×4, and 8×8 addition modes in the storage portion inside the radiation imaging apparatus 100.

In step S118, since the accumulation image generation processing is completed, the control portion 109 transfers a control command informing the end of image generation in the current correction image generation mode to the signal processing portion 101 via the control interface 110.

In step S119, the control portion 109 reads out N 1×1 accumulation images from the storage portion, averages the readout images, and saves the resultant image as a 1×1 correction image in the storage portion.

In step S120, the control portion 109 reads out N 2×2 accumulation images from the storage portion, averages the readout images, and saves the resultant image as a 2×2 correction image in the storage portion.

In step S121, the control portion 109 reads out N 4×4 accumulation images from the storage portion, averages the readout images, and saves the resultant image as a 4×4 correction image in the storage portion.

In step S122, the control portion 109 reads out N 8×8 accumulation images from the storage portion, averages the readout images, and saves the resultant image as an 8×8 correction image in the storage portion.

With the above operation, the generation of 1×1, 2×2, 4×4, and 8×8 correction images is completed.

In step S123, the control portion 109 notifies the signal processing portion 101 via the control interface 110 that the generation of correction images is completed. If it is necessary to generate correction images under control mode setting conditions different from those set in step S102, the control portion 109 repeats the processing from step S102. In contrast to this, if the generation of correction images is completed, the control portion 109 sets the imaging panel 105 in the sleep state in step S124 and terminates the correction image generation processing.

The above is the method of generating accumulation images in a plurality of addition modes by one time of accumulation and generating a correction image by averaging the images obtained by repeating the generation. When generating a correction image for gain correction in this correction image generation procedure, the control portion 109 may perform irradiation with a predetermined dose of X-rays during the accumulation period T of the imaging panel 105 in the X-ray irradiation processing in step S108. In contrast to this, when generating a correction image for offset correction, the control portion 109 may accumulate images without irradiation during the accumulation period T of the imaging panel 105.

In the example of the flowcharts shown in FIGS. 6A and 6B, the radiation imaging apparatus 100 saves the accumulation images obtained in steps S109 to S115 and generates correction images in steps S119 to S122. However, the generation of correction images is not limited to the specific generation portion and may be performed by, for example, the signal processing portion 101 that performs system control. In this case, the accumulation images generated in steps S109 to S115 are sequentially transferred to the signal processing portion 101 via the image data interface 111.

In this embodiment, in the above image signal readout operation, image signals are read out while the pixel addition mode is sequentially switched to the 1×1, 2×2, 4×4, and 8×8 pixel addition modes (sequentially enlarged). In contrast to this, when image signals are read out while the pixel addition mode is sequentially switched to the 8×8, 4×4, 2×2, and 1×1 pixel addition modes (sequentially reduced), the accumulation signal S1 and the noise signal S3 need to be sampled again every time the pixel addition mode is switched to restore the accumulation signal S1 and the noise signal S3 obtained by averaging in pixel addition processing.

Owing to the structure of the exemplary circuit of the pixel P, when the clamp signal PCL is activated to re-sample the noise signal S3, the clamp voltage VCL as a predetermined potential is input to the output terminal n2 of the clamp capacitor Cc1. This makes it impossible to read signals based on charges subsequently generated and accumulated in the photodiode PD. Accordingly, in this embodiment, it is necessary to read out signals over a plurality of times while performing pixel addition in the direction to sequentially enlarge the addition region instead of the direction to sequentially reduce the addition region.

As described above, according to the above embodiment, it is possible to read out accumulation signals held in the holding portion by imaging of one frame while sequentially switching the pixel addition mode from high resolution to low resolution and obtain correction images in a plurality of pixel addition modes by one time of imaging. This makes it possible to improve the efficiency of the generation of correction images.

### Second Embodiment

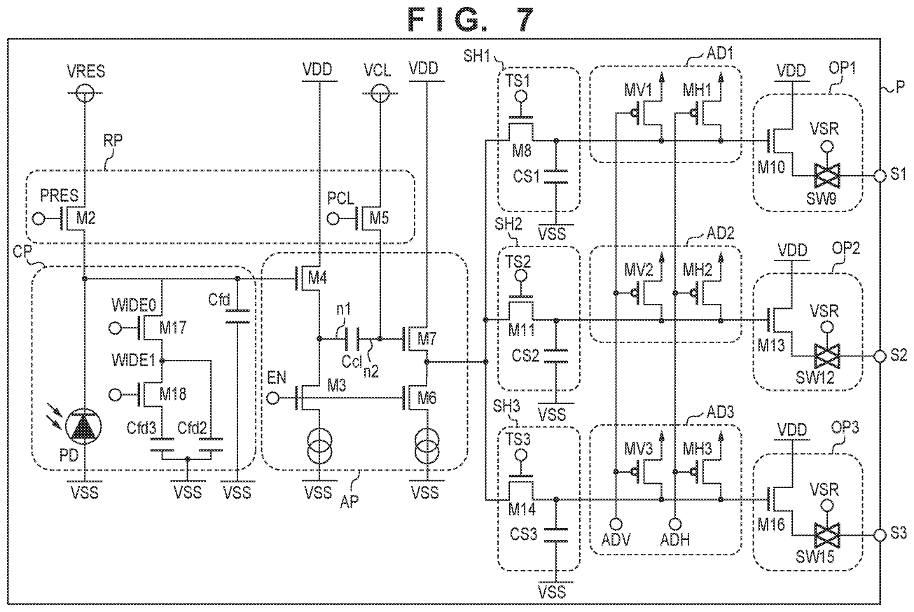

The hardware arrangement of a radiation imaging apparatus 100 according to the second embodiment will be described with reference to FIG. 7. The same reference numerals as in the first embodiment denote the same parts, and the difference between the first embodiment and the second embodiment will be mainly described below. FIG. 7 is an exemplary circuit diagram for explaining the schematic circuit of one pixel P in the radiation imaging apparatus 100 according to the second embodiment.

A conversion portion CP includes a photodiode PD, a transistor M17, a transistor M18, an FD capacitor Cfd, and additional capacitors Cfd2 and Cfd3 for sensitivity switching. Charges are generated in the photodiode PD in an amount corresponding to radiation, and the voltage of the FD capacitor Cfd which corresponds to the generated amount of charges is output to an amplification portion AP. In addition, the capacitor Cfd2 for sensitivity switching is used to switch the sensitivity of the pixel P with respect to radiation and is connected to the photodiode PD via the transistor M17 (switching element). When a sensitivity switching control signal WIDE0 is activated, the transistor M17 changes to the conductive state, and the voltage of the synthetic capacitor of the FD capacitor Cfd and the capacitor Cfd2 is output to the amplification portion AP. The capacitor Cfd3 is connected to the photodiode PD via the transistor M17 and the transistor M18. When the sensitivity switching control signals WIDE0 and WIDE1 are activated, the transistor M17 and the transistor M18 change to the conductive state, and the voltage of the synthetic capacitor of the FD capacitors Cfd, Cfd2, and Cfd3 is output to the amplification portion AP. That is, controlling the conductive states of the transistor M17 and the transistor M18 will output one of the following signals: a high-sensitivity signal that is a voltage corresponding to the charges converted by a high-sensitivity conversion portion CP, an intermediate-sensitivity signal that is a voltage corresponding to the charges converted by an intermediate-sensitivity conversion portion CP, and a low-sensitivity signal that is a voltage corresponding to the charges converted by a low-sensitivity conversion portion CP.

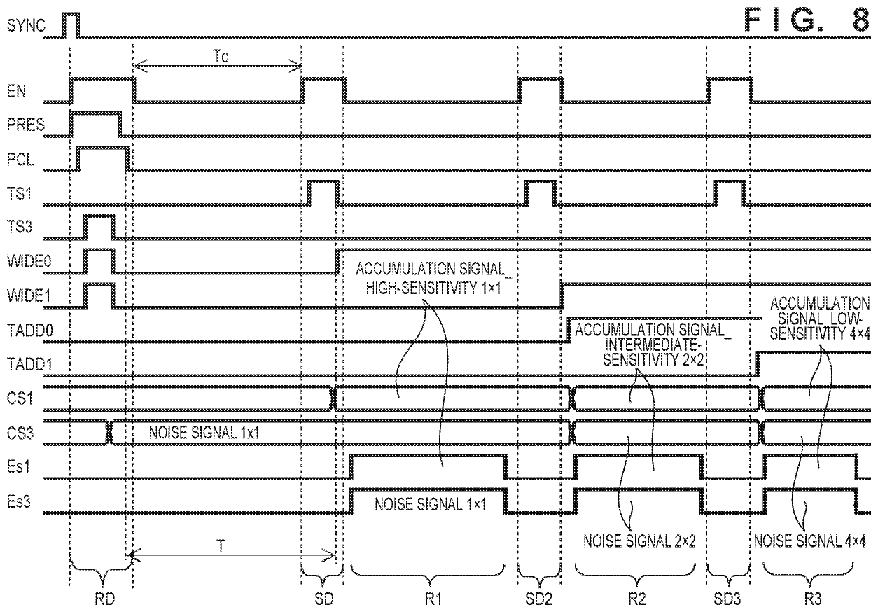

A method of driving the radiation imaging apparatus 100 according to the second embodiment will be described with reference to FIG. 8. The difference between the first embodiment and the second embodiment will be mainly described below. FIG. 8 is a timing chart showing an example of a method of continuously reading out correction images different in sensitivity and pixel addition count which are obtained by one time of accumulation while sequentially switching the sensitivity and the resolution from the high sensitivity and the high resolution to the low sensitivity and the low resolution.

In the driving method shown in FIG. 8, like the first embodiment, SYNC represents an external synchronization signal.

Reset driving RD will be described. Upon detecting the leading edge of a pulse of the signal SYNC, a control portion 109 starts driving for the generation of a frame image. The control portion 109 activates an enable signal EN, a reset signal PRES, and sensitivity switching control signals WIDE0 and WIDE1. This sets the transistor M17 and the transistor M18 in the conductive state and resets the charges in the photodiode PD, the floating diffusion capacitor Cfd, and the sensitivity switching additional capacitors Cfd2 and Cdf3 of the conversion portion CP. As a result, a voltage corresponding to a voltage from the conversion portion CP at the time of reset is input to an input terminal n1 of a clamp capacitor Cc1. The control portion 109 then activates a clamp signal PCL. This inputs a clamp voltage VCL to an output terminal n2 of the clamp capacitor Cc1. A control signal TS3 is temporarily activated until the clamp signal PCL is inactivated. With this operation, the clamp voltage VCL is transferred to and held in, as a noise signal, a holding capacitor CS3. Upon inactivating the control signal TS3, the control portion 109 inactivates the reset signal PRES and then inactivates the clamp signal PCL. This starts an accumulation period T. The control portion 109 inactivates the enable signal EN. This ends the reset driving RD.

Sample hold driving SD1 will be described next. The control portion 109 activates the enable signal EN after the lapse of a period Tc since the inactivation of the enable signal EN in the reset driving RD. The control portion 109 then temporarily activates a control signal TS1 while keeping the sensitivity switching control signals WIDE0 and WIDE1 inactive. A high-sensitivity accumulation signal S1 accumulated in the accumulation period T is transferred to and held in a holding capacitor CS1 (that is, the high-sensitivity accumulation signal is sampled). Upon completing the sampling, the control portion 109 activates the sensitivity switching control signal WIDE0. Activating the sensitivity switching control signal WIDE0 will set the transistor M17 in the conductive state and make the capacitor of the conversion portion CP become the synthetic capacitor of the FD capacitor Cfd and the FD capacitor Cfd2. The output of the conversion portion CP becomes the intermediate-sensitivity voltage of the synthetic capacitor of the FD capacitors Cfd and Cfd2 which corresponds to the charges generated by the photodiode PD during the accumulation period T. The control portion 109 inactivates the enable signal EN. This ends the sample hold driving SD1.

At the time of the end of the sample hold driving SD1, the addition mode is set to the 1×1 addition mode with both TADD0 and TADD1 being at “Low”, and the high-sensitivity accumulation signal S1 and a noise signal S3 are respectively held in the holding capacitor CS1 and a holding capacitor CS3 while each pixel is independently separated. Upon completion of the sample hold driving SD1, the control portion 109 maintains the state of the 1×1 addition mode, that is, starts reading out an image from the pixels in the entire imaging region while TADD0 and TADD1 are kept at “Low”.

Sample hold driving SD2 will be described next. The control portion 109 activates the enable signal EN after the lapse of the readout period R1 for a high-sensitivity 1×1 accumulation signal. The control portion 109 then temporarily activates the control signal TS1 while keeping the sensitivity switching control signal WIDE0 active. The intermediate-sensitivity accumulation signal S1 accumulated in this manner is transferred to and held in the holding capacitor CS1 (that is, the intermediate-sensitivity accumulation signal is sampled). Upon completion of the sampling, the control portion 109 activates the sensitivity switching control signal WIDE1. Activating the sensitivity switching control signals WIDE0 and WIDE1 will set the transistors M17 and M18 in the conductive state and make the capacitor of the conversion portion CP become the synthetic capacitor of the FD capacitor Cfd and the capacitors Cfd2 and Cfd3. The output of the conversion portion CP becomes the low-sensitivity voltage of the synthetic capacitor of the FD capacitors Cfd and the capacitors Cfd2 and Cfd3 which corresponds to the charges generated by the photodiode PD during the period T. The control portion 109 inactivates the enable signal EN. This ends the sample hold driving SD2.

Upon completion of the sample hold driving SD2, the control portion 109 switches TADD0 from “Low” to “High” to switch the pixel addition mode to the 2×2 pixel addition mode. The control portion 109 generates a 2×2 intermediate-sensitivity accumulation signal in the period R2 by sequentially switching the vertical scanning circuit 403 and the horizontal scanning circuit 404 for each two pixels (a predetermined count) in accordance with the 2×2 pixel addition state.

Sample hold driving SD3 will be described next. The control portion 109 activates the enable signal EN after the lapse of the readout period R2 for an intermediate-sensitivity 2×2 accumulation signal. The control portion 109 then temporarily activates the control signal TS1 while keeping the sensitivity switching control signals WIDE0 and WIDE1 active. The intermediate-sensitivity accumulation signal S1 accumulated in this manner is transferred to and held in the holding capacitor CS1 (that is, the intermediate-sensitivity accumulation signal is sampled). With this operation, the sample hold driving SD3 is completed.

Upon completion of the sample hold driving SD3, the control portion 109 switches TADD1 from “Low” to “High” to switch the pixel addition mode to the 4×4 pixel addition mode. The control portion 109 generates a 4×4 low-sensitivity accumulation signal in the period R3 by sequentially switching the vertical scanning circuit 403 and the horizontal scanning circuit 404 for each four pixels (a predetermined count) in accordance with the 4×4 pixel addition state.

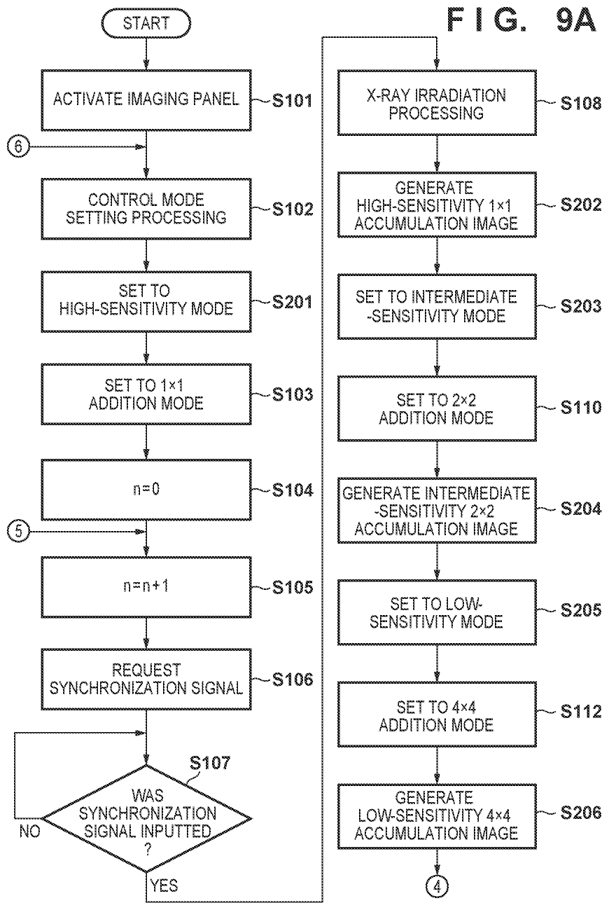

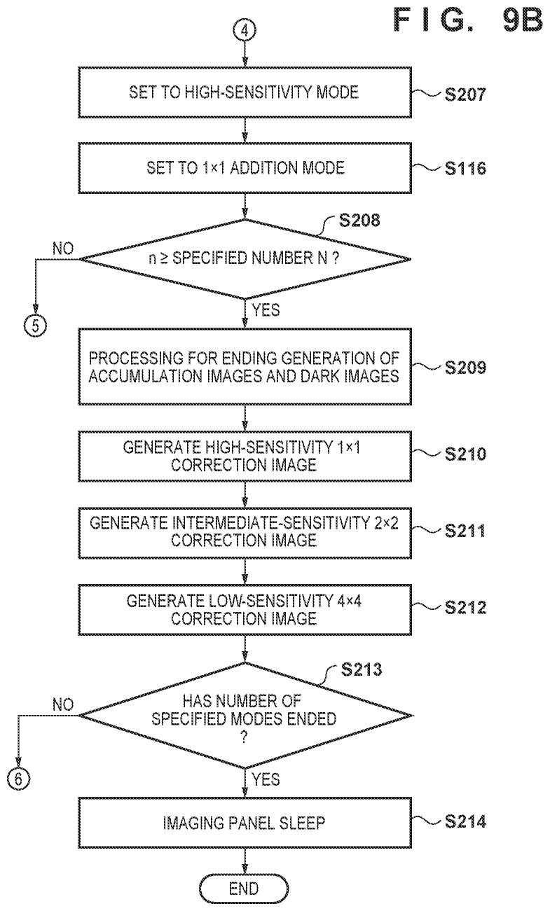

FIGS. 9A and 9B are flowcharts showing an example of processing from the accumulation of images in synchronism with a synchronization signal by the radiation imaging apparatus 100 to the generation of correction images in the different sensitivity modes and pixel addition modes, namely, the high-sensitivity 1×1, intermediate-sensitivity 2×2, and low-sensitivity 4×4 modes, based on the accumulation images. The same step numbers denote steps in which the same processes as those in the first embodiment are performed, and the difference between the first embodiment and the second embodiment will be mainly described below.

Steps S101 and S102 are the same as those in the first embodiment.

In step S201, the control portion 109 sets the sensitivity mode of a pixel array 120 of an imaging panel 105 to the high-sensitivity mode as a default mode.

Steps S103 to S108 are the same as those in the first embodiment.

In step S202, the control portion 109 reads out a high-sensitivity 1×1 accumulation signal, generates a high-sensitivity 1×1 accumulation image, and saves the generated accumulation image in the radiation imaging apparatus 100 (not shown).

In step S203, the control portion 109 sets the sensitivity mode of the pixel array 120 of the imaging panel 105 to the intermediate-sensitivity mode.

Step S110 is the same as that in the first embodiment.

In step S204, the control portion 109 reads out an intermediate-sensitivity 2×2 accumulation signal, generates an intermediate-sensitivity 2×2 accumulation image, and saves the generated accumulation image in the radiation imaging apparatus 100 (not shown).

In step S205, the control portion 109 sets the sensitivity mode of the pixel array 120 of the imaging panel 105 to the low-sensitivity mode.

Step S112 is the same as that in the first embodiment.

In step S206, the control portion 109 reads out a low-sensitivity 4×4 accumulation signal, generates a low-sensitivity 4×4 accumulation image, and saves the generated accumulation image in the radiation imaging apparatus 100 (not shown).

In step S207, the control portion 109 sets the sensitivity mode of the pixel array 120 of the imaging panel 105 to the high-sensitivity mode as the default mode.

Step S116 is the same as in the first embodiment.

In step S208, the control portion 109 determines whether the processing of generating accumulation images in different sensitivity modes and different addition modes is completed a specified number N of times set in the control mode setting processing in step S102. If the generation is completed the specified number of times, processing in step S209 is executed. If the specified number of times has not been reached, the processing of generating an accumulation image is repeated from step S105.

When the accumulation image generation processing in steps S101 to S208 is completed, the N accumulation images in different sensitivity modes and different addition modes, including the high-sensitivity 1×1 mode, the intermediate-sensitivity 2×2 mode, and the low-sensitivity 4×4 mode, are stored in the storage portion in the radiation imaging apparatus 100.

In step S209, upon completing the accumulation image generation processing, the control portion 109 transfers a control command informing the end of the image generation in the current correction image generation mode to the signal processing portion 101 via the control interface 110.

In step S210, the control portion 109 reads out N high-sensitivity 1×1 accumulation images from the storage portion, performs averaging processing of the images, and saves the resultant image as a high-sensitivity 1×1 correction image in the storage portion.

In step S211, the control portion 109 reads out N intermediate-sensitivity 2×2 accumulation images from the storage portion, performs averaging processing of the images, and saves the resultant image as an intermediate-sensitivity 2×2 correction image in the storage portion.

In step S212, the control portion 109 reads out N low-sensitivity 4×4 accumulation images from the storage portion, performs averaging processing of the images, and saves the resultant image as a low-sensitivity 4×4 correction image in the storage portion.

With the above operation, the generation of the high-sensitivity 1×1, intermediate-sensitivity 2×2, and low-sensitivity 4×4 correction images is completed.

In step S213, the control portion 109 notifies the signal processing portion 101 via the control interface 110 that the generation of correction images is completed. If it is necessary to generate correction images under conditions different from the control mode setting conditions set in step S102, the processing from step S102 is repeated. In contrast to this, if the generation of correction images is completed, the imaging panel 105 is set in the sleep state in step S214, thus completing the correction image generation.

The above is the method of generating accumulation images in a plurality of sensitivity modes and a plurality of addition modes by one time of accumulation and then generating a correction image by averaging the images obtained by repeating the generation. When a correction image for gain correction is to be generated in this correction image generation procedure, a specified dose of X-rays may be applied in the X-ray irradiation processing in step S108 during the accumulation period T of the imaging panel 105. In contrast to this, when a correction image for offset correction is to be generated, accumulation may be performed without irradiation during the accumulation period T of the imaging panel 105.

In the example of the flowcharts shown in FIGS. 9A and 9B, the radiation imaging apparatus 100 saves the accumulation images in steps S202 to S206 and generates the correction images in steps S210 to S212. However, the correction image generation portion is not specifically limited. For example, the signal processing portion 101 that performs system control may perform the above correction image generation. In this case, the accumulation images generated in steps S202 to S206 are sequentially transferred to a signal processing portion 101 via an image data interface 111.

In this embodiment, in the above image signal readout operation, image signals are read out while the sensitivity mode is sequentially changed to the high-sensitivity, intermediate-sensitivity, and low-sensitivity modes. In contrast to this, when a readout operation is to be performed while the sensitivity mode is sequentially changed to the low-sensitivity, intermediate-sensitivity, and high-sensitivity modes, it is necessary (owing to the nature of charges) to make the photodiode PD generate the amount of charges again in accordance with radiation every time the sensitivity mode is switched to return the voltage of the synthetic capacitor of the FD capacitor Cfd, the capacitor Cfd2, and the capacitor Cfd3 to the initial voltage. Accordingly, in this embodiment, it is necessary to perform a readout operation a plurality of times while switching the sensitivity to lower sensitivities. In addition, as in the first embodiment, it is necessary to perform a readout operation a plurality of times while performing pixel addition in a direction to sequentially enlarge the pixel addition region instead of a direction to sequentially reduce the pixel addition region.

As has been described above, in the second embodiment described above, images are read out while accumulation signals obtained by imaging of one frame are sequentially switched in order of, for example, the high-sensitivity 1×1 mode, the intermediate-sensitivity 2×2 mode, and the low-sensitivity 4×4 mode. This makes it possible to obtain correction images in a plurality of sensitivity modes and a plurality of pixel addition modes by one time of imaging. This can improve the efficiency of the generation of correction images.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2021-206267, filed Dec. 20, 2021, and Japanese Patent Application No. 2022-155294, filed Sep. 28, 2022 which are hereby incorporated by reference herein in their entirety.

What is claimed is:

1. A radiation imaging apparatus comprising:
a plurality of pixels arranged in a matrix pattern, each pixel including a signal generation portion configured to generate a signal based on accumulated charges and a holding portion configured to hold a signal output from the signal generation portion;
a first switch configured to commonly connect a predetermined number of pixels of the plurality of pixels to add the signals from the predetermined number of pixels; and
a readout circuit configured to read out accumulation signals held in the holding portion over a plurality of times while changing a pixel addition region by using the first switch and performing pixel addition to obtain a plurality of images different in pixel addition count with respect to one time of charge accumulation.

2. The apparatus according to claim 1, wherein the readout circuit reads out accumulation signals held in the holding portion over a plurality of times while performing pixel addition in a direction to sequentially enlarge the pixel addition region by using the first switch.

3. The apparatus according to claim 1, wherein the plurality of images different in pixel addition count are correction images.

4. The apparatus according to claim 1, further comprising a second switch configured to switch sensitivity and a readout circuit configured to obtain a plurality of images different in sensitivity with respect to one time of charge accumulation by holding and reading out accumulation signals output from the signal generation portion with respect to the holding portion over a plurality of times while switching sensitivity by using the second switch.

5. The apparatus according to claim 4, wherein the readout circuit holds and reads out accumulation signals output from the signal generation portion with respect to the holding portion over a plurality of times while switching the sensitivity to lower sensitivities by using the second switch.

6. The apparatus according to claim 4, wherein the plurality of images different in sensitivity are correction images.

7. The apparatus according to claim 1, wherein the plurality of pixels each include the plurality of holding portions.

8. The apparatus according to claim 7, wherein the plurality of pixels each include the three holding portions.

9. The apparatus according to claim 7, wherein one of the plurality of holding portions is a holding portion configured to hold an accumulation signal generated by the signal generation portion while radiation is received and another of the plurality of holding portions is a holding portion configured to hold a dark signal generated by the signal generation portion while no radiation is received.

10. The apparatus according to claim 9, further comprising a subtraction circuit configured to obtain a difference between the accumulation signal and the dark signal.

11. The apparatus according to claim 1, wherein the readout circuit obtains a plurality of images equal in pixel addition count.

12. The apparatus according to claim 11, further comprising a generation circuit configured to generate a correction image by averaging the plurality of images equal in pixel addition count.

13. The apparatus according to claim 1, wherein the first switch is a switch configured to connect adjacent pixels to each other.

14. A method of controlling a radiation imaging apparatus including a plurality of pixels arranged in a matrix pattern, each pixel including a signal generation portion configured to generate a signal based on accumulated charges and a holding portion configured to hold a signal output from the signal generation portion and a first switch configured to commonly connect a predetermined number of pixels of the plurality of pixels to add the signals from the predetermined number of pixels, the method comprising:
reading out accumulation signals held in the holding portion over a plurality of times while performing pixel addition in a direction to sequentially enlarge a pixel addition region by using the first switch to thereby obtain a plurality of images different in pixel addition count with respect to one time of charge accumulation.

15. The method according to claim 14, wherein the radiation imaging apparatus further comprises a second switch configured to switch sensitivity and obtains a plurality of images different in sensitivity with respect to one time of charge accumulation by holding and reading out accumulation signals output from the signal generation portion with respect to the holding portion over a plurality of times while switching the sensitivity to lower sensitivities by using the second switch.

16. A non-transitory computer-readable storage medium storing a program for executing a method of controlling a radiation imaging apparatus including a plurality of pixels arranged in a matrix pattern, each pixel including a signal generation portion configured to generate a signal based on accumulated charges and a holding portion configured to hold a signal output from the signal generation portion and a switch configured to commonly connect a predetermined number of pixels of the plurality of pixels to add the signals from the predetermined number of pixels, the method comprising:
reading out accumulation signals held in the holding portion over a plurality of times while performing pixel addition in a direction to sequentially enlarge a pixel addition region by using the switch to thereby obtain a plurality of images different in pixel addition count with respect to one time of charge accumulation.
